(19) United States
(12) Patent Application Publication
(10) Pub. No.: US 2021/0085811 A1
(43) Pub. Date: Mar. 25, 2021

(54) SYSTEMS AND METHODS FOR QUANTIFYING ULTRAVIOLET DOSAGES

(71) Applicant: Bolb Inc., Livermore, CA (US)

(72) Inventors: Peter Gordon, Livermore, CA (US); Ling Zhou, Dublin, CA (US); Jianping Zhang, Arcadia, CA (US); Ying Gao, Fremont, CA (US); Cuong Le, Livermore, CA (US)

(73) Assignee: Bolb Inc., Livermore, CA (US)

(21) Appl. No.: 17/020,862

(22) Filed: Sep. 15, 2020

Related U.S. Application Data
(60) Provisional application No. 62/905,843, filed on Sep. 25, 2019.

(51) Int. Cl. A61L 2/10

(52) U.S. Cl. CPC A61L 2/10; A61L 2300/606

(57) ABSTRACT

Apparatus for measuring a dosage of disinfection light. The apparatus includes: a measurement pad including a photochromic material that changes its color in response to a dosage of disinfection light incident on the measurement pad; and a dosage scale bar having a plurality of units that have different colors, wherein the dosage of the disinfection light is determined by comparing the color of the measurement pad with the different colors of the plurality of units.

### CROSS-REFERENCE TO PRIOR APPLICATIONS

This application claims priority of a U.S. Patent Application No. 62/905,843, Sep. 25, 2019, which is hereby incorporated by reference in its entirety.

### BACKGROUND

### A. Technical Field

The present invention relates to disinfection devices, and more particularly, to indicators for visually verifying the ultraviolet (UV) intensity or UV dosage delivered to the objects to be disinfected.

### B. Background of the Invention

Overuse of antibiotics, fungicides and other chemical disinfectants lead to antibiotic resistance for many pathogens. One promising alternative to chemical disinfection is photonic disinfection, which employs energetic photons, especially those in the UV spectral range to disrupt the DNA and RNA molecules and/or protein functions of the pathogen. Typically, the photonic sources are solid-state emitters, or light-emitting diodes (LEDs).

Unlike traditional UV sources, such as gas-discharge lamps which emit in both visual and the deep ultraviolet, LEDs have minimal visual emission. The lack of visual component in the light output from LEDs makes it difficult for equipment users to ascertain the intensity and distribution of light. As such, there is a need for indicators that can help UV LED equipment users to visually verify the coverage area and output intensity or the dosage delivered as a function of time at the targeted surface at any incident angle, under direct or indirect UV illumination.

Furthermore, for a conventional color-chaining indicator that changes its color as the dosage of UV light incident on the indicator changes, a calibration of the color change needs to be carried out specific to each discrete LEDs' spectral range in order to make the color change definable and useful to users. In general, a calibrated color change may take many forms and errors in interpreting the color change due to variability of human eyes' response to a color change and environmental lighting. As such, there is a need for a reliable approach for calibrating the color change so that the conventional errors in interpreting the color change may be reduced, corrected, or eliminated.

### SUMMARY OF THE DISCLOSURE

According to one aspect of the present invention, an apparatus for measuring a dosage of disinfection light includes: a measurement pad including a photochromic material that possesses a calibrated response of color change to a dosage of the disinfection light incident on the measurement pad; and a dosage scale bar having a plurality of units that have different colors, wherein the dosage of the disinfection light is determined by comparing a color of the measurement pad with the different colors of the plurality of units.

According to another aspect of the present invention, an apparatus for measuring a dosage of disinfection light includes: a pad; and a stenciled messaging system printed on the pad with a photochromic material, wherein the color of the pad is same as the color of the stenciled messaging system when the pad is not exposed to disinfection light and wherein the color of the stenciled messaging system becomes different from the color of the pad when a preset dosage of the disinfection light is incident on the pad to thereby reveal the stenciled messaging system.

According to another aspect of the present invention, an apparatus for measuring a dosage of disinfection light includes: one or more pills that include a surface formed of photochromic material that changes a color in response to a dosage of disinfection light incident on the pills.

According to another aspect of the present invention, a device for interpreting dosage information of disinfection light incident on a dosage indicator includes: a microprocessor; a camera for capturing an image of a dosage indicator that includes a measurement pad and an image of a light compensation card that includes a neutral grey color patch. The measurement pad may include a photochromic material that changes a color in response to a dosage of disinfection light incident on the measurement pad. The device further includes: an image processing unit for processing the image of the dosage indicator and the image of the light compensation card and identify the measurement pad and the neutral grey color patch; a colorimeter reader for reading the color of the measurement pad and the color of the neutral grey color patch and determining the dosage of disinfection light incident on the measurement pad; and a light compensation unit for compensating, based on the color of the neutral grey color patch, an environmental light that is incident on the dosage indicator when the camera takes the image of the dosage indicator.

### BRIEF DESCRIPTION OF THE DRAWINGS

The patent or application file contains at least one drawing executed in color, Copies of this patent or patent application publication with color drawing(s) will be provided by the Office upon request and payment of the necessary fee.

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 shows an exemplary indicator calibration with different UV wavelengths according to embodiments of the present disclosure.

FIG. 2 shows a top view of a UVC dosage reading label according to embodiments of the present disclosure.

FIG. 3 shows a top view of a UVC dosage reading label according to embodiments of the present disclosure.

FIG. 4A shows a cross sectional view of the UVC dosage reading label in FIG. 3, taken along the line 4-4, according to embodiments of the present disclosure.

FIGS. 4B-4D show a cross sectional views of UVC dosage reading labels according to embodiments of the present disclosure.

FIGS. 5A-5C show UVC dosage reading labels according to embodiments of the present disclosure.

FIGS. 6A-6C show UVC dosage reading labels according to embodiments of the present disclosure.

FIG. 7A shows a portable device that interprets the UVC dosage information on a UVC dosage reading label according to embodiments of the present disclosure.

FIG. 7B shows a device for interpreting the UVC dosage information on a UVC dosage reading label according to embodiments of the present disclosure.

FIG. 7C shows a light compensation card according to embodiments of the present disclosure.

FIGS. 8A and 8B show exemplary applications of a UVC dosage reading label according to embodiments of the present disclosure.

FIG. 9 shows UVC dosage indicators according to embodiments of the present disclosure.

FIG. 10 shows a UV transmission (UVT) system for controlling the UVC dosage delivered to fluid according to embodiments of the present disclosure.

FIG. 11 shows an exemplary application of a UVC dosage reading label according to embodiments of the present disclosure.

FIG. 12 shows a schematic diagram of a system for implementing one or more aspects of the present disclosure.

### DETAILED DESCRIPTION OF THE PREFERRED EMBODIMENTS

In the following description, for the purposes of explanation, specific details are set forth in order to provide an understanding of the invention. It will be apparent, however, to one skilled in the art that the invention can be practiced without these details. One skilled in the art will recognize that embodiments of the present invention, described below, may be performed in a variety of ways and using a variety of means. Those skilled in the art will also recognize additional modifications, applications, and embodiments are within the scope thereof, as are additional fields in which the invention may provide utility. Accordingly, the embodiments described below are illustrative of specific embodiments of the invention and are meant to avoid obscuring the invention.

A reference in the specification to “one embodiment” or “an embodiment” means that a particular feature, structure, characteristic, or function described in connection with the embodiment is included in at least one embodiment of the invention. The appearance of the phrase “in one embodiment,” “in an embodiment,” or the like in various places in the specification are not necessarily all referring to the same embodiment.

For the purpose of illustration, the light is assumed to be UVC (wavelength range of 200-280 nm) in the following sections, even though the light in other wavelength ranges may also be used depending on the types of pathogens to be sterilized, or other benefits to be realized such as dermatological applications of UVB and curing applications of UVA, UVB and UVC. Likewise, the disinfection light source is assumed to be LED, even though other types of light sources may be used. In embodiments, an indicator system may be used to help UV LED equipment users to visually verify the coverage area, output intensity or the dosage delivered as a function of time at the targeted surface at any incident angle, under direct or indirect UV illumination.

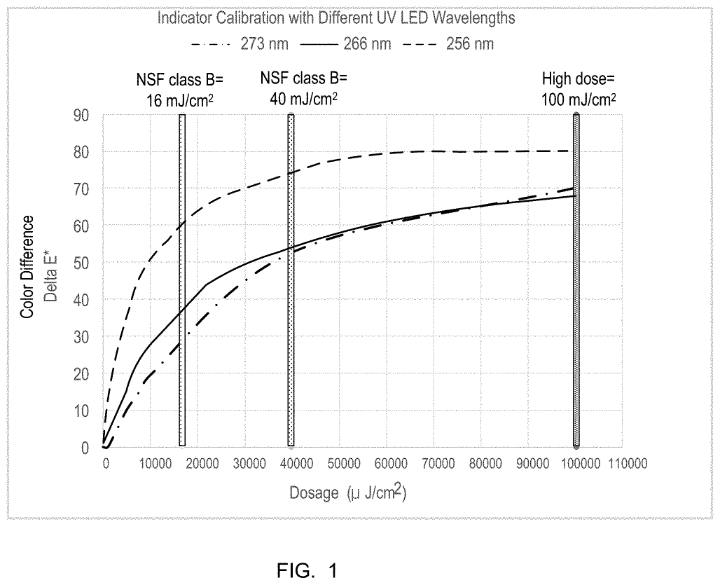

FIG. 1 shows an exemplary indicator calibration with different UV wavelengths according to embodiments of the present disclosure. As depicted, each curve in FIG. 1 represents a plot of color differences, delta-E (as defined by the CIE L*, a*, b* color coordinate system, for instance), as a function of light dosage delivered to an indicator. In FIG. 1, the delta-E (dE) in the y-axis was calculated according to delta E* 2000, and a dE of 1.0 is the smallest color difference that the human eye can perceive. In embodiments, the chemistry of the indicators is such that the wavelengths covered by UV LEDs, especially those with peak wavelengths between 260-315 nm, may result in readable color differences from 0 to at least 100 mJ/cm² on the indicator. In embodiments, the color difference may have dE value of at least 1 for every 10 mJ/cm². In FIG. 1, the National Science Foundation (NSF) class A (40 mJ/cm²) and class B (16 mJ/cm²) are the standards that represent the UV dosages required to deactivate various bacteria pathogens.

As depicted in FIG. 1, the color change (dE) curves, can be divided into two groups, one is for emissions of 260-280 nm, another is for sub-260 nm emissions. Due to stronger surface absorption of shorter wavelengths, dE is more significant under the same dosage for sub-260 nm emission is than for 260-280 nm, even though this does not mean that sub-260 nm emissions have better disinfection efficiency.

In embodiments, each indicator (which is also referred to as label) may include photochromic material that is designed to produce color shift in response to UV lights, where the UV lights may be generated by UV LEDs and include single or combinations of two or more dominant wavelengths that are illuminated either simultaneously or sequentially.

In embodiments, the indicators (or labels) may be capable of showing color shifts perceptible to human eyes, with dE of at least 10 (or any other suitable value) between unexposed and fully exposed (saturated) indicator color. In embodiments, each indicator may be exposed through either one-shot, or multi-shots accumulated overtime, and the UV light source may be operated in either continuous-wave or pulsed exposure mode.

TABLE 1
Color change per dosage of UV light in various
and dosage ranges and wavelengths

| | Color change (dE) | Accumulated |
|---|---|---|
| Wavelength of | per Dosage of UV | dosage range |
| UV light (nm) | light (per mJ/cm²) | (mJ/cm²) |
| 256 | 5.0 | 0-10 |
| | 0.73 | 10-40 |
| | 0.1 | 40-100 |
| 266 | 2.8 | 0-10 |
| | 0.8 | 10-40 |
| | 0.27 | 40-100 |
| 273 | 2.0 | 0-10 |
| | 1.0 | 10-40 |
| | 0.33 | 40-100 |

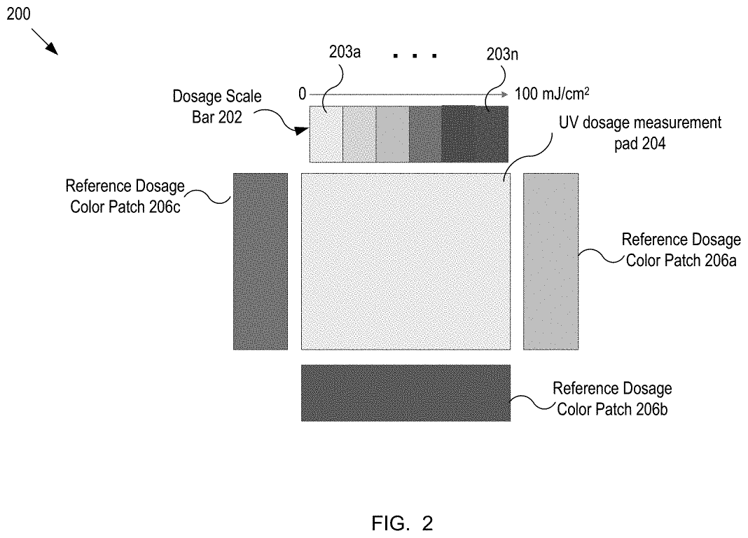

Table 1 shows a calibrated response of color change per dosage of UV light incident on a measurement pad (such as 204 in FIG. 2) in various dosage ranges and wavelengths. As shown in Table 1, the middle column represents a color change (dE) per dosage of UV light (i.e., the slope of each curve in FIG. 1) and the third column refers to the ranges of accumulated dosage incident on the measurement pad. In embodiments, the response of color change may depend on the wavelength of the disinfection light and the accumulated dosage of the disinfection light.

In embodiments, the measurement pads described in conjunction with FIGS. 2-11 may be calibrated in the manner described in conjunction with FIG. 1 (and Table 1). In embodiments, each of the measurement pad may change its color in a calibrated response to a dosage of disinfection light of a pre-defined narrow spectrum with full-width-half-maximum value of 12 nano meters or narrower incident on the measurement pad.

FIG. 2 shows a top view of a UVC dosage reading label (or indicator) 200 according to embodiments of the present disclosure. As depicted, the UVC dosage reading label 200 may have a dosage scale bar 202 having a plurality of units 203a-203n of different colors, where any two neighboring units may be of color difference 10 or higher. In embodiments, the UVC dosage reading label 200 may also include a UV dosage measurement pad 204 and three reference dosage color patches/bars 206a-206c. In embodiments, the colors in the reference dosage color patches/bars 206a-206c may be calibrated to respectively correspond to UVC dosages of 16, 40 and 100 mJ/cm² (NSF class B/A).

In embodiments, the reference dosage color patches 206a-206c may be neutral references for accurate color reading in any lighting with color cast. It is noted that the UVC dosage reading label 200 may include other suitable number of reference dosage color patches that each correspond to a reference UVC dosage. It is also noted that the color unit 203n may correspond to the maximum dosage of UV light, such as 100 mJ/cm², and the number of the color units 203a-203n may be varied to adjust the color difference (dE) between two neighboring color units.

In embodiments, the UV dosage measurement pad 204 may be infused with photochromic material. In embodiments, the UV dosage measurement pad 204 may have the same color as the color units 203a when UV dosage measurement pad 204 is not exposed to any UV light, and the UV dosage measurement pad 204 changes its color in response to the total UV dosage incident thereon. The user may compare the color of the UV dosage measurement pad 204 to the color units 203a-203n to determine the total UV dosage delivered to the UV dosage measurement pad 204. Also, in embodiments, the user may compare the color of the UV dosage measurement pad 204 to one (e.g. 206a) of the reference dosage color patches 206a-206c so as to determine whether the total dosage has reached the reference dosage (e.g. 16 mJ/cm²) of the patch.

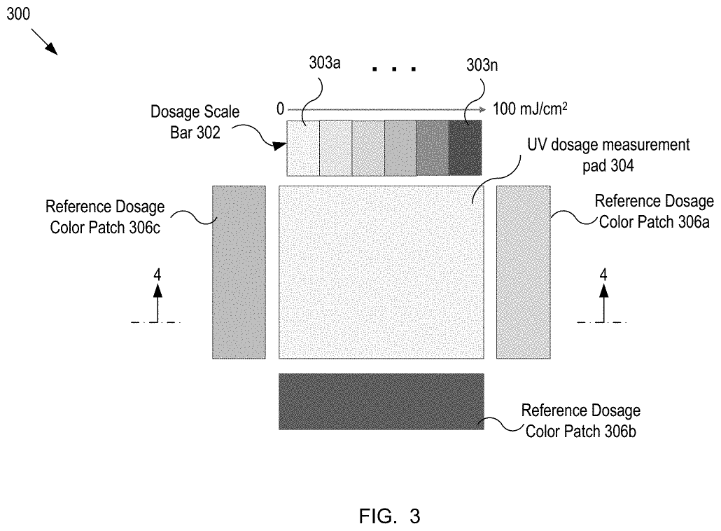

FIG. 3 shows a top view of a UVC dosage reading label 300 according to embodiments of the present disclosure. As depicted, the UVC dosage reading label 300 may have a dosage scale bar 302 that includes color units 303a-303n, a UV dosage measurement pad 304, and three reference dosage color bars/patches 306a-306c, where the three reference dosage color 306a-306c are calibrated to respectively correspond to UV dosages sufficient for 3-log killing rates for three different pathogens. It is noted that the UVC dosage reading label 300 may include other suitable number of reference dosage color patches that may correspond to UVC dosages sufficient for disinfecting other various pathogens of interest, respectively. In embodiments, the dosage scale bar 302 may be similar to the scale bar 202, and the user may compare the color of the UV dosage measurement pad 304 to the color units 303a-303n to determine the total UV dosage delivered to the UV dosage measurement pad 304. Likewise, the UV measurement pad 304 may be similar to the UV measurement pad 204, i.e., the UV dosage measurement pad 304 may be infused with photochromic material so that the UV dosage measurement pad 304 changes its color in response to the total dosage of the UV light incident thereon.

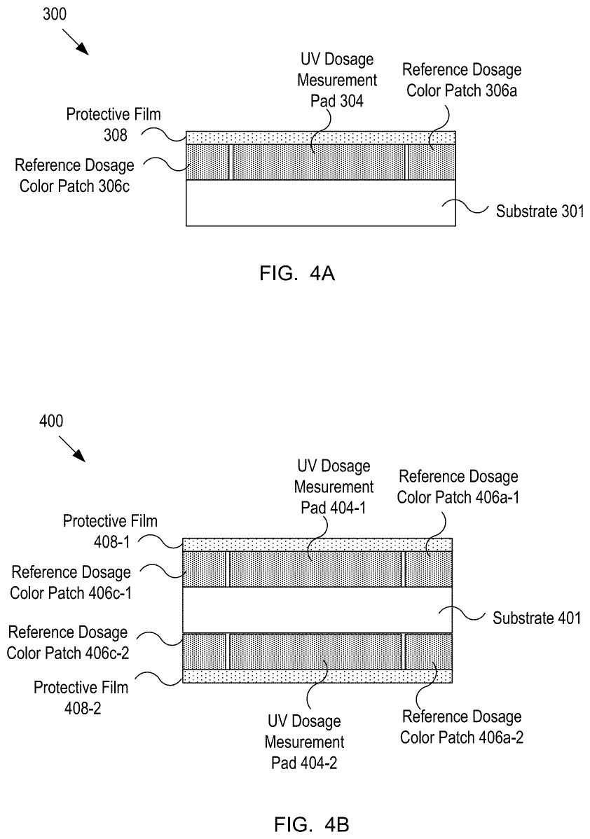

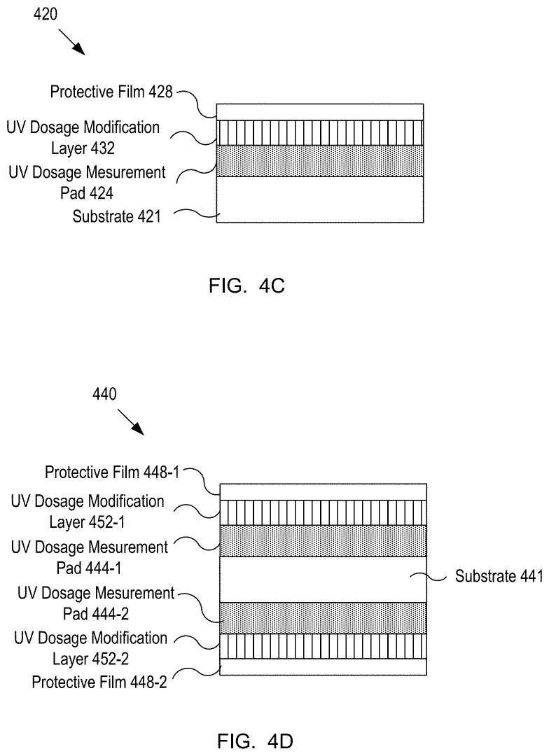

FIG. 4A shows a cross sectional view of the UVC dosage reading label 300 in FIG. 3, taken along the line 4-4, according to embodiments of the present disclosure. As depicted, the label 300 may include: a substrate 301; the UV dosage measurement pad 304 disposed on the substrate 301; the reference dosage color patches 306a-306c disposed on the substrate 301; the dosage scale bar 302 (not shown in FIG. 4A) disposed on the substrate 301; and a protective film 308 disposed on the UV dosage measurement pad 304, reference dosage color patches 306a-306c and dosage scale bar 302. In embodiments, the protective film 308 may be transparent to the UV light and protect the components of the UVC dosage reading label 300 against dust and/or moisture ablation. In alternative embodiments, the protective film 308 may be disposed on the UV measurement pad 304 only. It is noted that the UVC dosage reading label 200 may include a protection layer that is similar to the protection film 308.

In embodiments, the substrate 301 may be formed of material, such as Polytetrafluoroethylene (PTFE), Fluorinated ethylene propylene (FEP), TOPAS® cyclic-olefin-copolymer (COC) medical polymer, plexiglass, ceramic, or plastic, that provides mechanical strength for other components of the UVC dosage reading label 300. In embodiments, the UV dosage measurement pad 304, reference dosage color patches 306a-306c, dosage scale bar 302 and protective film 308 may be printed on the substrate 301 by suitable printing techniques.

FIG. 4B shows a cross sectional view of a UVC dosage reading label 400 according to embodiments of the present disclosure. As depicted, the UVC dosage reading label 400 may be double sided and include: a substrate 401; a pair of protective films 408-1 and 408-2; a pair of UV dosage measurement pads 404-1 and 404-2; a pair of first reference dosage color patches 406a-1 and 406a-2; a pair of second reference dosage color patches (not shown in FIG. 4B); a pair of third reference dosage color patches 406c-1 and 406c-2; and a pair of dosage scale bars (not shown in FIG. 4B) that are similar to the dosage scale bar 302. Each component of the UVC dosage reading label 400 may be formed of the same material as the corresponding component of the UVC dosage reading label 300. For instance, the protective films 408-1 and 408-2 may be formed of the same material as the protective film 308. In another example, each of the pair of dosage scale bars may be similar to the dosage scale bar 302.

In embodiments, a UVC dosage reading label may include a UV dosage measurement pad that begins to change its color only at a specific amount of dosage, such as a dosage of 16, 40 or 100 mJ/cm² (NSF class B/A or according to other accepted standards). In embodiments, such an effect may be achieved with an overlay coating on the indicator. FIG. 4C shows a cross sectional view of a UVC dosage reading label 420 according to embodiments of the present disclosure. As depicted, the UVC dosage reading label 420 may include: a substrate 421; a UV dosage measurement pad 424; a UV dosage modification (or attenuation) layer (film) 432 disposed on the UV dosage measurement pad 424; and a protective film 428 disposed on the UV modification layer 432.

In embodiments, the protective film 428 and UV dosage measurement pad 424 may be similar to the protective film 308 and the UV dosage measurement pad 304, respectively. In embodiments, the UV dosage modification layer 432 may attenuate the UV light to a desired degree, to thereby delay the onset of color shift/change in the UV dosage measurement pad 424 to a specific dosage, i.e., the UV dosage modification layer 432 may be used to control the UV sensitivity and partially absorb the ultraviolet light while substantially transparent to visible light. It is noted that, when the UV dosage modification layer 432 is included in the label 420, there may no longer be the need for color reference patches on the label 420 since the color shift only occurs above a specific dosage.

FIG. 4D shows a cross sectional view of a UVC dosage reading label 440 according to embodiments of the present disclosure. As depicted, the UVC dosage reading label 440 may be double sided and include: a substrate 441; a pair of protective films 448-1 and 448-2; a pair of UV dosage modification layers 452-1 and 452-2; and a pair of UV dosage measurement pads 444-1 and 444-2. Each component of the UVC dosage reading label 440 may be formed of the same material as the corresponding component of the UVC dosage reading label 420. For instance, each of the protective films 448-1 and 448-2 may be formed of the same material as the protective film 428.

In embodiments, another approach to make a UV dosage measurement pad (such as 402, 444-1 and 444-2) change its color only at a specific amount of dosage may be desensitizing the UV dosage measurement pad, i.e., the photochromic material in the UV dosage measurement pad may be desensitized so as to achieve the effect of delaying the normal color shift to a higher specific dosage set by the particular application requirement. In such a case, the UV dosage modification layers 432, 452-1 and 452-2 may not be necessary.

In embodiments, the protective film 428 (448-1 and 448-2) may be combined with the underlying UV dosage modification layer 432 (452-1 and 452-2), i.e., the protective film 428 (448-1 and 448-2) may be formed of material that not only attenuates the UV light to a desired degree but also protects the underlying UV dosage measurement pad 424 (444-1 and 444-2). In such a case, the dosage calibration of the UV measurement pad 424 (444-1 and 444-2) may be performed while the protective film 428 (448-1 and 448-2) is mounted thereon.

Referring back to FIGS. 4A and 4B, the protective film 308 (408-1 and 408-2) may be formed of material that is transparent to the UV light. However, if the material is not completely transparent so as to slightly attenuate the UV light, the dosage calibration of the UV measurement pad 304 (404-1 and 404-2) may be performed while the protective film protective film 308 (408-1 and 408-2) is mounted thereon.

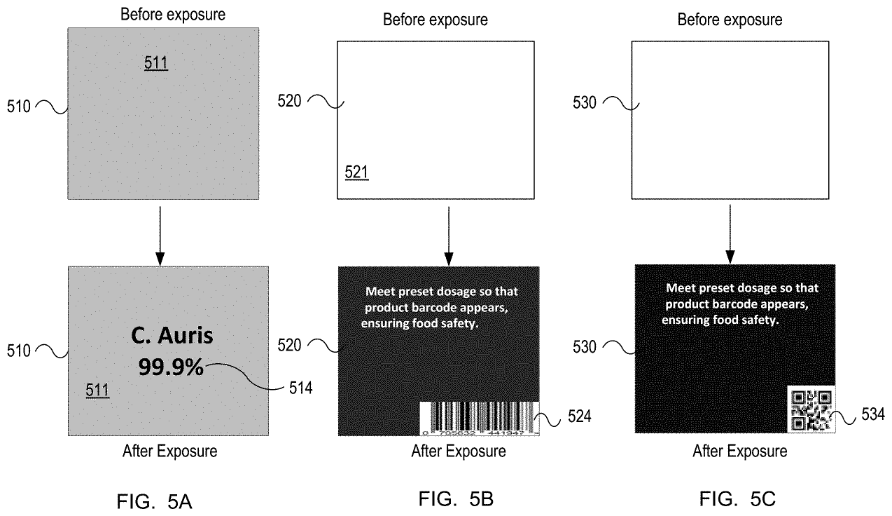

In embodiments, a UVC dosage reading label (or indicator) may include a stenciled communication and/or a status messaging system (or collectively stenciled messaging system) that uses contrasting colors to display one or more messages. In embodiments, the message may take the form of either natural language or symbols readable by human operators or machines. FIGS. 5A-5C show UVC dosage reading labels according to embodiments of the present disclosure. As depicted in FIG. 5A, a UVC dosage reading label 510 may include a stenciled messaging system that reveals the hidden message 514 “C Auris 99.9%” when the total dosage delivered to the UVC dosage reading label 510 reaches a preset dosage.

In embodiments, the stenciled messaging system in the UVC dosage reading label 510 may include a partially desensitized photochromic layer, i.e., the stenciled messaging system may be the hidden message 514 printed with partially desensitized photochromic material. The photochromic material diffused in the stenciled messaging system may be partially desensitized so as to achieve the effect of delaying the normal color shift to a higher specific dosage set by a particular application requirement. In embodiments, the background pad 511 may not include any photochromic material so that its color remains unchanged in response to the disinfection light. In embodiments, the stenciled messaging system in the UVC dosage reading label 510 may have the same color as the background pad 511 until the specific UV dosage is delivered to the UVC dosage reading label 510.

As depicted in FIG. 513, a UVC dosage reading label 520 may include a stenciled messaging system that reveals a hidden text message “Meet preset dosage . . . safety” and a hidden bar code 524 when the total dosage delivered to the UVC dosage reading label 520 reaches a preset dosage. In embodiments, the stenciled messaging system in UVC dosage reading label 520 may include a UV attenuation layer (or masking layer) disposed on a UV dosage measurement pad (background) 521. For instance, the stenciled messaging system may be in the form of the hidden message and barcode printed with UV attenuation material. In embodiments, the UV dosage measurement pad 521 may change its color when the total UV dosage delivered to the UVC dosage reading label 520 reaches a preset value, while the color of the stenciled messaging system covered by the UV attenuation layer remains unchanged to reveal the hidden message and bar code 524.

In embodiments, the degree of UV transparency of the UV attenuation layer that covers the stenciled messaging system may be controlled through variations, such as thickness of the UV attenuation layer, or the degree of desensitization of the photochromic material of the background 521, for the purpose of controlling the dosage necessarily accumulated prior to revealing the stenciled messages.

In alternative embodiments, the stenciled messaging system of the UVC dosage reading label 520 may have photochromic material that is different from the photochromic material of the UV dosage measurement pad (background) 521, and the color difference between the stenciled messaging system and the UV dosage measurement pad (background) 521 can be perceived when the total dosage of UV delivered to the UVC dosage reading label 520 exceeds a specific level.

As shown in FIG. 5C, the UVC dosage reading label 530 may reveal various hidden messages and symbols upon receiving a specific dosage of UV light. In embodiments, the symbol the UVC dosage reading label 530 may take the form of a text message and a quick response (QR) code 534. It is noted that the symbol in a UVC dosage reading label may take other suitable form of proprietary coding systems. In embodiments, the symbols may be decoded by various hand-held equipment, such as laser scanners or smart phones.

In one use case scenario of the present invention, during transportation or processing of the food items, the stenciled labels may be affixed to the food product being transported or processed, and the hidden messages can only be seen after a pre-determined UV dosage has been accumulated, such as 16 mJ/cm², or any dosage value deemed important for that particular application. Logistical workers equipped with a scanner or a smart phone or other equipment which may properly read, interpret, record and re-transmit the messages may confirm proper UV treatment of the food items being transported or processed, only after the food item has been exposed to the dosages required. This step helps ensure the biosecurity of the transportation or processing chain, and any subsequent process steps will not be allowed to proceed unless certain disinfection steps have been performed. In other embodiments, the hidden messages may only be revealed after multiple exposures at different wavelengths. The absence of exposure on the indicators may indicate low UV leakage, i.e., the equipment is effective in containing the UV light within and the environment is safe for personnel to operate without wearing any protection.

In embodiments, the method of use for stenciled labels may be modified according to different use cases, including but not limited to: food or packaging items on a conveyor belt; medical equipment or dirty surfaces to be cleaned in a medical facility; handrails in public transport equipment; likely pathogen-contaminated areas in public spaces including schools or restaurants. Such indicators may also be in a tethered form and allowed to float through air or water to be disinfected.

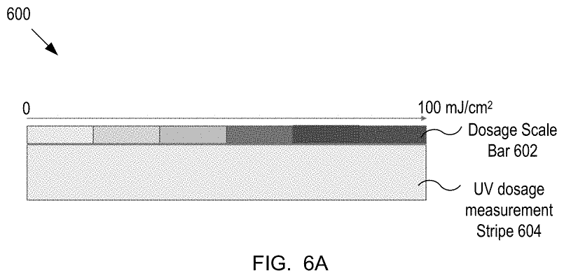

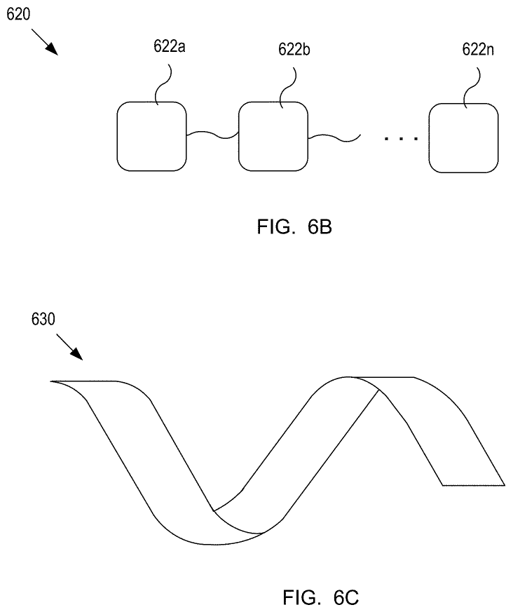

FIG. 6A shows a UVC dosage reading label 600 according to embodiments of the present disclosure. As depicted, the UVC dosage reading label 600 may include a dosage scale bar 602 and a UV dosage measurement strip 604 that is similar to the UV dosage measurement pad 204. The UVC dosage measurement strip 604 may be wrapped inside or outside a tube and can be used to either ascertain the UV dosage received, or lack thereof to ensure personnel UV safety.

FIG. 6B shows a UVC dosage reading label 620 according to embodiments of the present disclosure. As depicted, the UVC dosage reading label 620 may include daisy-chained UVC dosage reading labels 622a-622n, where each of the UVC dosage reading labels 622a-622n may be similar to the UVC dosage reading labels in FIGS. 2-5C. In alternative embodiments, one or more of the UVC dosage reading labels 622a-622n may be similar to the UV dosage measurement pad 204 (or 304). In embodiments, the UVC dosage reading label 620 may be used in curing or food production line environments as part of direct exposure to objects traveling down the line or conveyor belt.

FIG. 6C shows a UVC dosage reading label 630 according to embodiments of the present disclosure. As depicted, the UVC dosage reading label 630 may have the shape of a conical or helical strip and be similar to the UVC dosage reading label 600 or UV dosage measurement pad 204 (or 304). In embodiments, the UVC dosage reading label 630 may be simply an indicator that can be wrapped around a cylindrical or spherical surface to measure the dosage delivered to the curved surface.

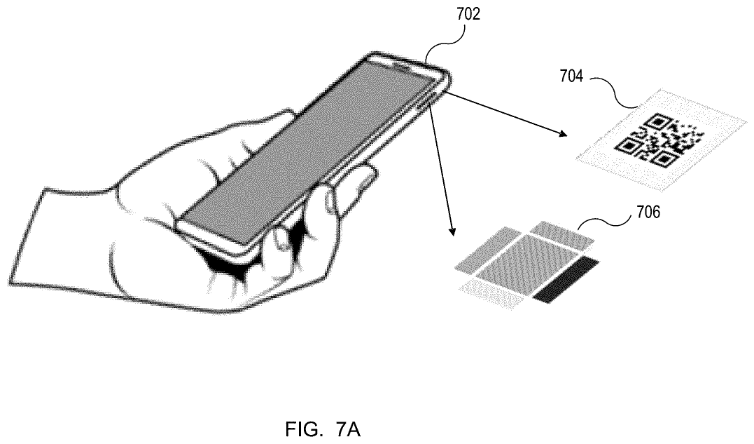

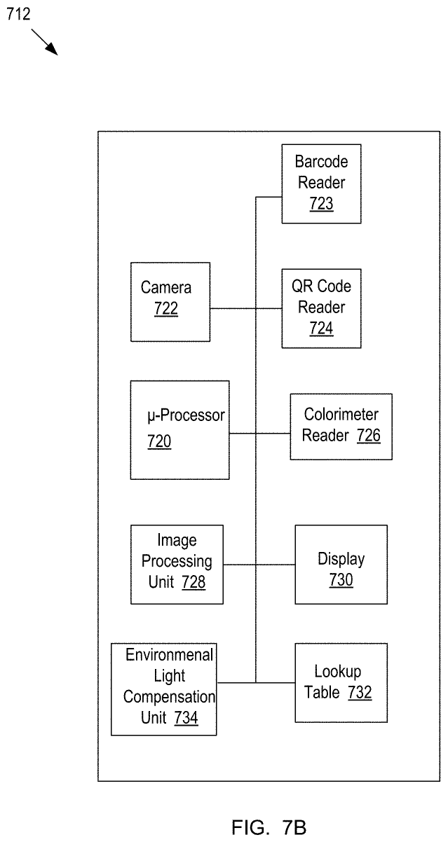

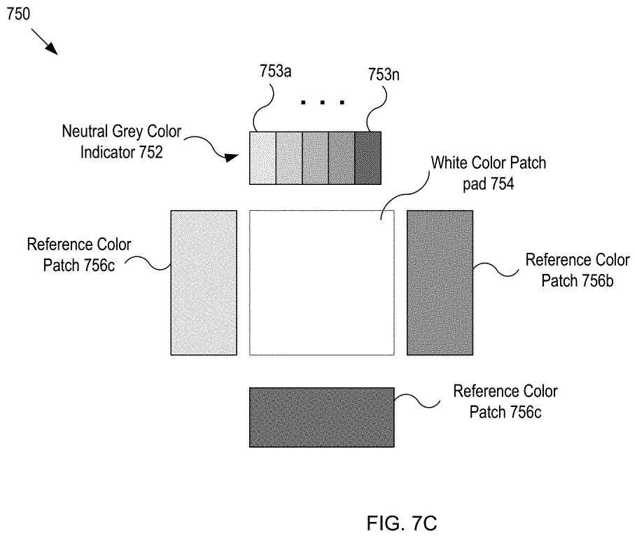

FIG. 7A shows a smart phone 702 that interprets the UVC dosage information on a UVC dosage reading label 704 and/or 706 according to embodiments of the present disclosure. It is noted that other suitable types of portable devices having cameras may be used in place of the smart phone. As depicted, an application installed in the smart phone 702 may interpret the aforementioned messages using images captured by a photographic system, such as the phone's own camera or camera attachment. In embodiments, the smart phone 702 may take a photo of the UVC dosage reading label and scan the image of the UVC dosage reading label to recognize the components of the UVC dosage reading label.

In embodiments, the smart phone 702 may capture the image of a QR code revealed by the UVC dosage reading label 704, where the UVC dosage reading label 704 may be similar to the label 534 in FIG. 5C. In embodiments, the smart phone 702 may capture the image of a UVC dosage reading label 706 that may be similar to the UVC dosage reading label 200 (or 300). In embodiments, the application installed in the smart phone 702 may interpret the color shift of the UVC dosage measurement pad (such as 204) and display the UV dosage delivered to the UVC dosage reading label.

In embodiments, the smart phone application 702 may take advantage of the reference color patches, interpret and display the label. In embodiments, the smart phone may take advantage of the neutral gray color patch or other reference color patches on the indicator, to nullify the effect of any color cast in the environmental lighting. In embodiments, the smart phone may display various information, such as color coordinates of the exposed patch, dosages corresponding to the LED wavelength, and percentages and species of pathogens killed, by referencing to a database. In embodiments, such measurement results may be uploaded or transmitted wirelessly to another database or display unit remotely. In embodiments, such measurements may be used to remotely control the UV LED lighting units to effect changes in output.

FIG. 7B shows a device 712 (such as the smartphone 702) for interpreting the UVC dosage information on a UVC dosage reading label (or equivalently indicator) according to embodiments of the present disclosure. In embodiments, each component of the device 712 may be a hardware, a software, a firmware or any combination thereof. As depicted, the device 712 may include: a μ-processor 702 coupled to and operating various components of the device; a camera 722 for capturing an image of a UVC dosage reading label in FIGS. 2-6C, such as 704 and 706; an image processing unit 728 for processing the captured images and identifying the components of the UVC dosage reading label; a barcode reader 723 for reading and interpreting the barcode in the UVC dosage reading label; a QR code reader 724 for reading and interpreting the QR code in the UVC dosage reading label 704; a colorimeter reader 726 for converting the color of the measurement pad (such as 204) into the UV dosage incident on the measurement pad; a display 730 for displaying images/messages thereon and operating as a graphic user interface (GUI); an environmental light compensation unit (or shortly, light compensation unit) 734 for compensating the environmental light incident on the UVC dosage reading label; and a lookup table 732. In embodiments, the lookup table 732 may be arranged as color-dosage pairs. In alternative embodiments, the lookup table 732 may be located outside the device 712 and accessed by the device 712 via a wireless communication channel.

In embodiments, the UVC dosage reading label may include the dosage scale bar 202 and UV dosage measurement pad 204, and the image processing unit 728 may process the captured image of the UVC dosage reading label to identify these components. Then, the colorimeter reader 726 may read the color of the UV dosage measurement pad 204 and the colors of the units 203a-203n of the dosage scale bar 202, and compare the color of the UV dosage measurement pad 204 to the colors of the units 203a-203n of the dosage scale bar 202, and, based on the comparison, determine the dosage of UV light incident on the UV dosage measurement pad 204, and display the UV dosage information on the display 730. In alternative embodiments, the colorimeter reader 726 may read the color of the UV dosage measurement pad 204 and retrieve a closest one of the plurality of color-dosage pairs in the lookup table 732 to the color of the measurement pad so as to determine the dosage of UV light incident of the measurement pad.

In embodiments, the QR code reader 726 may read the QR code (such as 534) and display the hidden message associated with QR code on the display 730. In embodiments, the barcode reader 723 may read the barcode (such as 524) and display the hidden message associated with barcode on the display 730.

When an image of a UVC dosage reading label is captured by the camera 722, the environmental light incident on the UVC dosage reading labels may be added to the image, causing uncertainty in reading the color of the label and negatively impacting the color interpretation. In embodiments, a light compensation card may be used to compensate the effect of the environmental light on color interpretation. FIG. 7C shows a light compensation card 750 according to embodiments of the present disclosure. As depicted, the light compensation card 750 may include one or more of: a neutral grey color indicator 752; a white color patch 754; and one or more reference color patches 756a-756c. In embodiments, the neutral grey color indicator 752 may include one or more neutral grey color patches 753a-753n that each have a grey color with a preset reflectance across the visual spectrum. In embodiments, the reference color patches 756a-756c may be primary colors of a color space, such as RGB or CIELAB. However, it should be apparent to those of ordinary skill in the art that other suitable number of reference color patches may be included in the light compensation card 750. In embodiments, the light compensation card 750 and a UVC dosage reading label in FIGS. 2-6C may be located side by side, i.e., the light compensation card 750 and a UVC dosage reading label in FIGS. 2-6C may be printed on the same side of a substrate. In alternative embodiments, the light compensation card 750 may be printed on a substrate separate from the UVC dosage reading labels in FIGS. 2-6C. In yet another embodiment, the light compensation card 750 may include only one grey color patch and conform to a standard color card, such as a neutral grey card sold under the name Kodak R-27 with 18% reflectance by the Eastman Kodak Company located in Rochester, N.Y.

In embodiments, the device 712 may read the colors of the components 752, 754 and 756a-756c of the light compensation card 750 in the similar manner as the colors of the components of the UVC dosage reading labels in FIGS. 2-6C are read, i.e., the camera 722 may capture an image of the light compensation card 750, the image processing unit 728 may identify the components 752, 754 and 756a-756c of the light compensation card 750, and the colorimeter reader 726 may read the colors of the components 752, 754 and 756a-756c. Then, based on the colors of the components 752, 754 and 756a-756c, the environmental light compensation unit 734 may compensate the contribution of the environmental light to the colors of the components of the UVC dosage reading labels in FIGS. 2-6C, to thereby eliminate the color cast of environmental light.

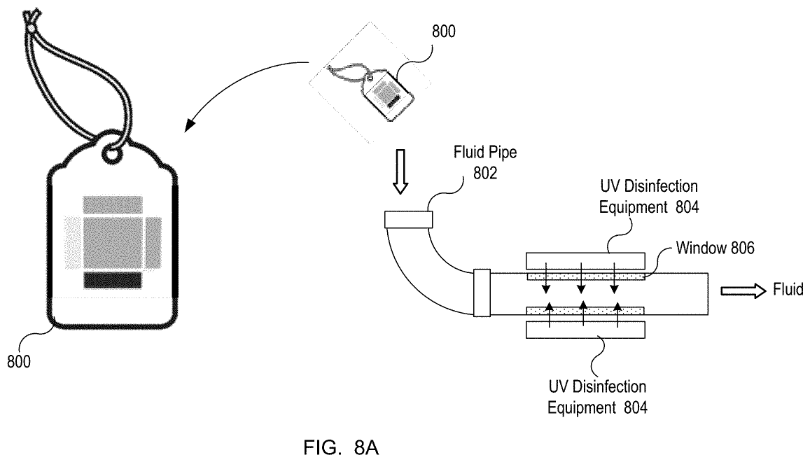

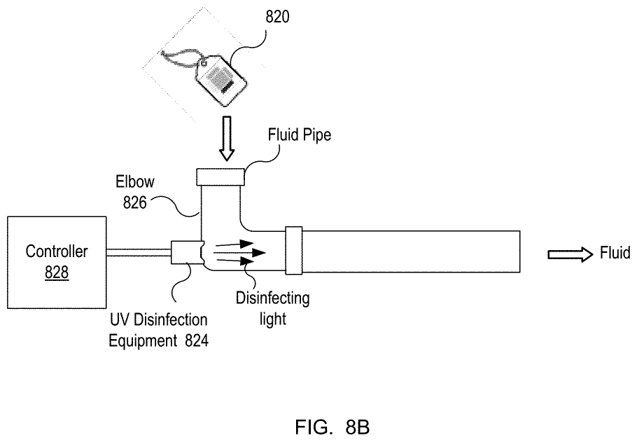

FIG. 8A shows an exemplary application of a UVC dosage reading label (or UVC dosage indicator) 800 according to embodiments of the present disclosure. As depicted, the UVC dosage indicator 800, which may be similar to the labels in FIGS. 2-6C, may be attached to a string. In embodiments, the indicator may be inserted into or wrapped outside a fluid pipe 802, where either air or water stream may carry the indicator 800 toward the exit of the fluid pipe 802. As the fluid flows through the pipe, the fluid may be disinfected by the UVC light that was emitted by UVC disinfection equipment 804 through the windows 806. After a preset duration of time, the indicator 800 may be retrieved via the string, and the indicator 800 may be interpreted by human operator, or a smart phone, or a colorimeter so as to determine the dosage of UVC light delivered to the indicator 800. Also, based on the dosage of UVC light delivered to the indicator 800, the amount of the dosage of UVC light delivered to the fluid may be determined.

It is noted that other suitable light source may be used to deliver UV light to the fluid in a fluid pipe. FIG. 8B shows an exemplary application of a UVC dosage reading label (or UVC dosage indicator) 820 according to embodiments of the present disclosure. As depicted, the UV disinfection equipment (or equivalently disinfection device) 824 may be installed in an elbow 826 and coupled to a controller 828 that controls the operation of the UV disinfection equipment 824. The indicator 820 may be used in the similar manner as the indicator 800 to determine the amount of the dosage of UVC light delivered to the fluid. More detailed description of the UV disinfection equipment 824 may be found in the copending U.S. patent application Ser. No. 15/808,904, entitled “Flowing Fluid Disinfectors and Submersible UV Light Device,” the entire disclosure of which, except for any inconsistencies, is incorporated herein by reference.

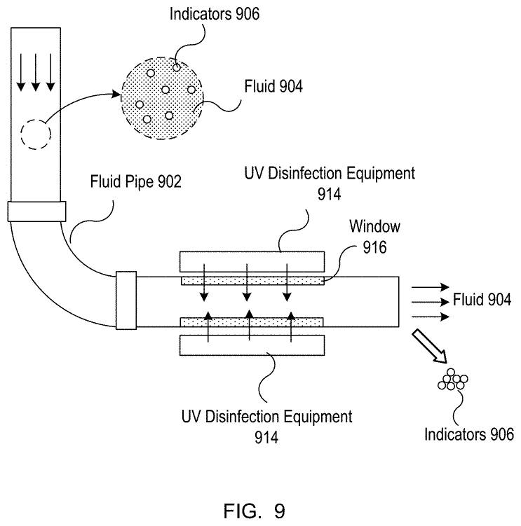

FIG. 9 shows UVC dosage indicators 906 according to embodiments of the present disclosure. In embodiments, the UVC dosage indicators 906 may be pills that are round in shape and dimensioned to immerse, float and sustain in fluid 904 to be disinfected. In embodiments, the UVC dosage indicators 906 may be formed of or coated with photochromic material. In embodiments, the fluid 904 may be water or air, and the UVC dosage indicators 906 may be balls of diameters of 1-10 mm.

In alternative embodiments, the UVC dosage indicators 906 may be small particles with an average diameter of any other suitable range. For instance, the UVC dosage indicators 906 may be microspheres that simulate a floating aerosol or droplets in an airstream or flowing organisms in a water stream and may be formed of or coated with photochromic material. In embodiments, the average diameter of the UVC dosage indicators 906 may be in any other suitable range so that the indicators can be seen under microscope or more readily and immediately by human eye.

In embodiments, the UVC dosage indicators 906 may be used to determine the dosage of the UV light delivered to the fluid 904. For instance, one or more UVC dosage indicators 906 in the fluid 904 may be inserted at the entrance end in the duct, conduit, tube, or pipe 902, and exposed to the UV light that was emitted by UVC disinfection equipment 914 through the windows 916. Upon passing through the section where the UVC dosage indicators 906 are exposed to the UV light, the UVC dosage indicators 906 may be separated from the fluid 904 and the color change of the UVC dosage indicators 906 may be measured to determine the amount of UV light delivered to the UVC dosage indicators 906 (and to the fluid 904). It is noted that the other suitable light source, such as the UV disinfection equipment 824, may be used to deliver UV light to the fluid 904.

In embodiments, the device 712 may interpret the UVC dosage information on the UVC dosage indicators 906 in the similar manner as interpreting the UVC dosage information on the UVC dosage reading labels in FIGS. 2-6C. For instance, the device 712 may take an image of the indicators 906 separated from the fluid, identify the indicators in the image, and read the color of the indicators in the image to determine the amount of UV light delivered to the indicators 906 (and to the fluid 904). For instance, the device 712 may retrieve a closest one of the plurality of color-dosage pairs in the lookup table 732 to the color of the indicators 906 so as to determine the dosage of UV light incident on the indicators.

In embodiments, an indicator system may include a colorimeter that may carry a standard illuminator which generates light with known color coordinates, may read an exposed indicator, and may display the UV dosages by comparing the color difference between unexposed and exposed indicators. In embodiments, such dosage measurements may be used to remotely control the UV LED lighting units to sense and effect changes in output.

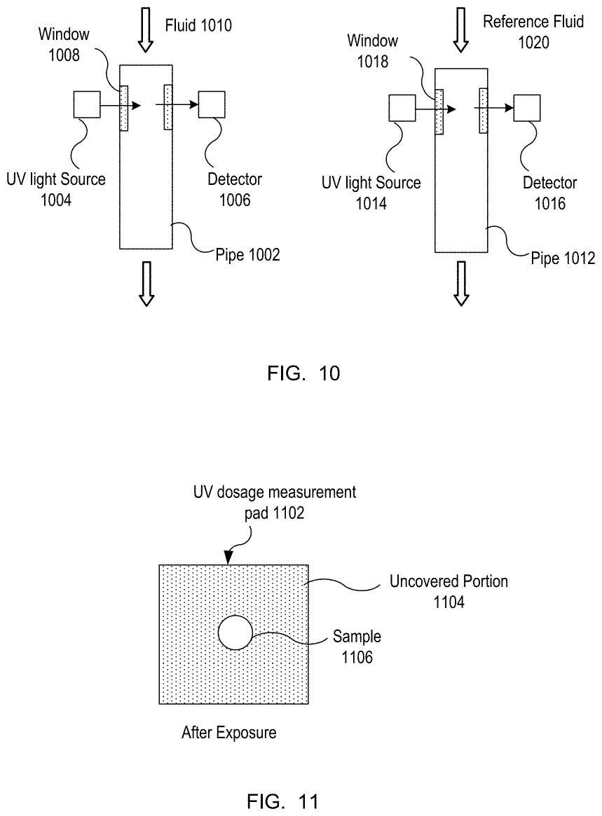

FIG. 10 shows a UV transmission (UVT) system for controlling the UVC dosage delivered to fluid according to embodiments of the present disclosure. As depicted, the UV light source 1004 (or 1014) may emit UV light of known intensity through the window 1008 (or 1018), and the detector 1006 (or 1016) may measure the light transmitted through the fluid 1010 (or 1020) that flows through the pipe 1002 (or 1012). In embodiments, the pipe 1012, UV light source 1014, detector 1016 and window 1018 may be identical to the pipe 1002, UV light source 1004, detector 1006 and window 1008, respectively.

In embodiments, the fluid 1010 may be the fluid to be disinfected while the reference fluid 1020 may be distilled water. The intensity of the UV light detected by the detector 1006 may be compared to the intensity of the UV light detected by the detector 1004, and the compared intensities may be used to control the UV disinfection equipment 914 to sense and recognize the absorption and effect source output changes as a response to attenuation of light transiting the fluid to be disinfected 1010.

Currently, the UVC disinfection industry does not know how much an aerosol or droplet attenuates the germicidal UV light transiting to its core, and this lack of information may create inactivation uncertainty. In embodiments, the dose indicators in FIGS. 2-6C may be used to determine the UV transmittance (at, for example, 272 nm) of a pathogen laden sample, such as an inoculate of various shapes, diameters, and constitutions, dry or wet, heavy bovine serum or light, other tissue, mucus, or blood. FIG. 11 shows an exemplary application of a UVC dosage reading label according to embodiments of the present disclosure. As depicted, the UV dosage measurement pad 1102, which may be similar to the UV dosage measurement pad 204 (or 304), may be calibrated to change its color in response to the UV dosage delivered thereto. In embodiments, a drop of pathogen laden sample (or shortly sample) 1106 may be plopped on the UV dosage measurement pad 1102, and the UV dosage measurement pad 1102 may be exposed to the UV light. As the sample 1106 may be opaque and absorb a portion of the UV light, the area covered by the sample 1106 may have a different color than the uncovered portion 1104 of the UV dosage measurement pad 1102. In embodiments, this color difference between the uncovered portion 1104 and the area covered by the sample 1106 may be used to determine the attenuation (or equivalently absorption) of the UV light by the sample. More specifically, the color difference may be used to determine the difference in the delivered intensity or dose of the UV light, which may be in turn used to determine attenuation and hence deduce the UV transmission (UVT) reduction that the sample 1106 produces.

In embodiments, the indicators (labels) in FIGS. 2-11 may be waterproof by a special coating/film and used in normal temperature ranges, such as from −25° C. to 65° C., for instance. In embodiments, the indicators may be reused by being heated to temperatures substantially above normal use temperatures, to thereby allow the color to fade back to the color prior to UV exposure. In embodiments, the temperatures required to fade or reset the color may be close to 100° C., in air on a hot surface, in heated steam, or in a hot liquid, such as water. In alternative embodiments, the temperatures to reset the indicator may be close to 150° C. or higher.

It is well known in the art that human UV exposure can lead to acute effects, such as erythema, photokeratitis or photoconjunctivitis, as well as long term effects, such as accelerated skin aging, basal cell carcinoma, squamous cell carcinoma, malignant melanoma or cataracts. The International Commission for Non-Ionizing Radiation Protection (ICNIRP) has published international recommendations for maximum UV-exposure levels. For the effective radiant exposure Heff (biologically weighted dose), the daily exposure (8 hours) limit value is 30 J/m² with an additional requirement that the unweighted UVA dose HUVA shall not exceed a daily exposure limit value of 104 J/m².

In embodiments, the UVC reading labels in FIGS. 2-6C may be used as an exposure indicator for worker safety dose verifications. For instance, the workers who may be exposed to UV light may wear the UV reading labels during their working hours in the same manner as the conference attendees wear their badges. In embodiments, each UVC reading label may be modified to have a dose trigger that changes its color when the UVC reading label's exposure has reached the maximum UV exposure levels set by the international Commission on Non-ionizing Radiation Protection (ICNIRP). In embodiments, the UVC reading label may be a nonwoven textile sewn or integrated into a personal protective equipment or clothes.

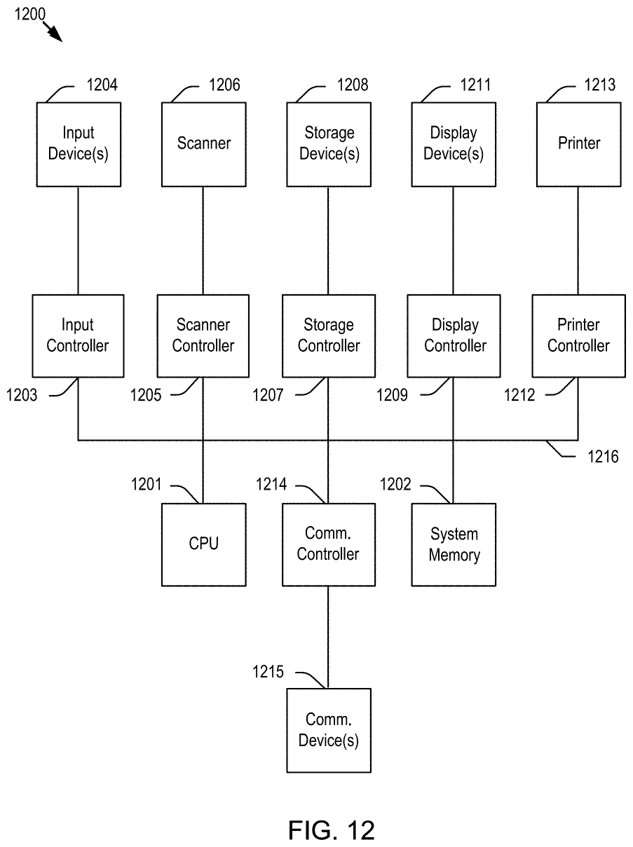

FIG. 12 shows a schematic diagram of a system 1200 for implementing one or more aspects of the present disclosure. It will be understood that the functionalities shown for system 1200 may operate to support various embodiments of the electronic devices (such as smartphone, etc.) for interpreting the UVC dosage information on a UVC dosage reading label in FIGS. 2-11—although it shall be understood that an electronic device may be differently configured and include different components. As illustrated in FIG. 12, system 1200 includes a central processing unit (CPU) 1201 that provides computing resources and controls the computer. CPU 1201 may be implemented with a microprocessor or the like, and may also include a graphics processor and/or a floating point coprocessor for mathematical computations. System 1200 may also include a system memory 1202, which may be in the form of random-access memory (RAM) and read-only memory (ROM).

A number of controllers and peripheral devices may also be provided, as shown in FIG. 12. An input controller 1203 represents an interface to various input device(s) 1204, such as a keyboard, mouse, or stylus. There may also be a scanner controller 1205, which communicates with a scanner 1206. System 1200 may also include a storage controller 1207 for interfacing with one or more storage devices 1208 each of which includes a storage medium such as magnetic tape or disk, or an optical medium that might be used to record programs of instructions for operating systems, utilities and applications which may include embodiments of programs that implement various aspects of the present invention. Storage device(s) 1208 may also be used to store processed data or data to be processed in accordance with the invention. System 1200 may also include a display controller 1209 for providing an interface to a display device 1211, which may be a cathode ray tube (CRT), a thin film transistor (TFT) display, or other type of display. System 1200 may also include a printer controller 1212 for communicating with a printer 1213. A communications controller 1214 may interface with one or more communication devices 1215, which enables system 1200 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 1216, which may represent more than one physical bus. However, various system components may or may not be in physical proximity to one another. For example, input data and/or output data may be remotely transmitted from one physical location to another. In addition, programs that implement various aspects of this invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

While the invention is susceptible to various modifications and alternative forms, specific examples thereof have been shown in the drawings and are herein described in detail. It should be understood, however, that the invention is not to be limited to the particular forms disclosed, but to the contrary, the invention is to cover all modifications, equivalents, and alternatives falling within the scope of the appended claims.

What is claimed is:

1. An apparatus for measuring a dosage of disinfection light, comprising:
a measurement pad including a photochromic material that possesses a calibrated response of color change to a dosage of the disinfection light incident on the measurement pad; and
a dosage scale bar having a plurality of units that have different colors,
wherein the dosage of the disinfection light is determined by comparing a color of the measurement pad with the different colors of the plurality of units.

2. The apparatus of claim 1, wherein the calibrated response of color change increases as a wavelength of the disinfection light decreases and wherein the response of color change decreases as the dosage of the disinfection light increases.

3. The apparatus of claim 2, wherein, when the wavelength of the disinfection light ranges from 250-280 nm, the calibrated response of color change ranges 2.0-5.0 per mJ/cm², 0.6-1.0 per mJ/cm² and 0.1-0.4 per mJ/cm² for the accumulated dosage of the disinfection light in ranges of 0-10 mJ/cm², 10-40 mJ/cm², and 40-100 mJ/cm², respectively.

4. The apparatus of claim 1, wherein two neighboring units of the plurality of units have a color difference of at least 10.

5. The apparatus of claim 1, further comprising:
at least one reference color patch having a color that corresponds to a reference dosage of the disinfection light incident on the measurement pad.

6. The apparatus of claim 1, further comprising:
a substrate, the measurement pad and the dosage scale bar being disposed on a top surface of the substrate; and
a protective film disposed on the measurement pad and formed of material that is transparent to the disinfection light.

7. The apparatus of claim 6, further comprising:
an attenuation film disposed between the measurement pad and the protective film and formed of material that attenuates the disinfection light.

8. The apparatus of claim 6, further comprising:
an additional measurement pad disposed on a bottom surface of the substrate; and
an additional dosage scale bar disposed on the bottom surface of the substrate.

9. The apparatus of claim 1, further comprising:
a stenciled messaging system printed on the measurement pad with a material that attenuates the disinfection light,
wherein the color of the measurement pad is same as a color of the stenciled messaging system when the measurement pad is not exposed to the disinfection light and
wherein the color of the measurement pad becomes different from the color of the stenciled messaging system when a preset dosage of the disinfection light is incident on the measurement pad to thereby reveal the stenciled messaging system.

10. The apparatus of claim 9, wherein the stenciled messaging system includes at least one of a message, a barcode and a quick response (QR) code.

11. An apparatus for measuring a dosage of disinfection light, comprising:
a pad; and
a stenciled messaging system printed on the pad with a photochromic material,
wherein the color of the pad is same as a color of the stenciled messaging system when the pad is not exposed to disinfection light and
wherein the color of the stenciled messaging system becomes different from the color of the pad when a preset dosage of the disinfection light is incident on the pad to thereby reveal the stenciled messaging system.

12. The apparatus of claim 11, wherein the stenciled messaging system includes at least one of a message, a bar code and a QR code.

13. An apparatus for measuring a dosage of disinfection light, comprising:
one or more pills that include a surface formed of photochromic material that changes a color in response to a dosage of disinfection light incident on the pills.

14. The apparatus of claim 13, wherein the one or more pills are round in shape and dimensioned to immerse, float and sustain in fluid,
wherein the color of the one or more pills is used to determine a dosage of the disinfection light delivered to the fluid.

15. A device for interpreting dosage information of disinfection light incident on a dosage indicator, comprising:
a microprocessor;
a camera for capturing an image of a dosage indicator that includes a measurement pad and an image of a light compensation card that includes a neutral grey color patch, the measurement pad including a photochromic material that changes a color in response to a dosage of disinfection light incident on the measurement pad;
an image processing unit for processing the image of the dosage indicator and the image of the light compensation card and identify the measurement pad and the neutral grey color patch;
a colorimeter reader for reading the color of the measurement pad and the color of the neutral grey color patch and determining the dosage of disinfection light incident on the measurement pad; and
a light compensation unit for compensating, based on the color of the neutral grey color patch, an environmental light that is incident on the dosage indicator when the camera takes the image of the dosage indicator.

16. The device of claim 15, wherein the light compensation card further includes a white color patch and a plurality of reference color patches and wherein the light compensation unit compensates the environmental light based on the color of the neutral grey color patch and one or more of the colors of the white color patch and the plurality of reference color patches

17. The device of claim 15, wherein the dosage indicator includes a dosage scale bar having a plurality of units that have different colors and wherein the colorimeter reader reads the different colors of the plurality of units and compares the color of the measurement pad to the different colors to determine the dosage of light incident on the measurement pad.

18. The device of claim 15, further comprising:
a lookup table having a plurality of color-dosage pairs,
wherein the colorimeter reader determines the dosage of light incident on the measurement pad by retrieving a closest one of the plurality of color-dosage pairs to the color of the measurement pad.

19. The device of claim 15, further comprising:
a barcode reader for reading a barcode in the dosage indicator and displaying a message associated with the barcode on the display.

20. The device of claim 15, further comprising:
a quick response (QR) code reader for reading a QR code in the dosage indicator and displaying a message associated with the QR code on the display.
